(19) United States
(12) Patent Application Publication
(10) Pub. No.: US 2025/0227410 A1
(43) Pub. Date: Jul. 10, 2025

(54) COMMUNICATION ASSEMBLIES FOR SURGICAL SYSTEMS

(71) Applicant: Intuitive Surgical Operations, Inc., Sunnyvale, CA (US)

(72) Inventors: Kirk Nangreaves, Campbell, CA (US); Catherine Porter, Santa Clara, CA (US); Mark Zimmer, Fremont, CA (US); Yong Mo Moon, San Ramon, CA (US); Christopher R. Burns, San Jose, CA (US); Christian Michael Lueke, Kiel (DE); Stephan Senkbeil, Kiel (DE); Robert F. Young, Santa Cruz, CA (US)

(21) Appl. No.: 19/014,461

(22) Filed: Jan. 9, 2025

Related U.S. Application Data
(60) Provisional application No. 63/619,581, filed on Jan. 10, 2024.

(51) Int. Cl. H04R 1/40; A61B 90/00; G02B 23/24; H04R 1/02; H04R 1/08; H04R 3/00

(52) U.S. Cl. CPC H04R 1/406; A61B 90/08; G02B 23/2461; H04R 1/028; H04R 1/086; H04R 3/005; H04R 2201/401

(57) ABSTRACT

A communication assembly of a medical system includes a cover plate and a circuit board spatially separated from the cover plate by a gap. One or more point light sources and a plurality of microphone sets are electrically coupled to the circuit board and disposed between the circuit board and the cover plate. A light guide is disposed at least partially between the circuit board and the cover plate and provides at least one light path within the gap to direct and diffuse light. A plurality of gaskets define an audio passageway extending across the gap from a first seal that seals to a microphone of one of the microphone sets to a second seal that seals around an audio opening defined in the cover plate.

### CROSS-REFERENCE TO RELATED APPLICATIONS

This application claims the priority benefit of U.S. Provisional Application No. 63/619,581, filed Jan. 10, 2024, which is hereby incorporated by reference herein in its entirety.

### FIELD

Disclosed embodiments relate to medical systems and, in particular, communication assemblies for surgical systems.

### BACKGROUND

Minimally invasive medical techniques (e.g., laparoscopy) have been used to reduce the amount of extraneous tissue which may be damaged during diagnostic or surgical procedures, thereby reducing patient recovery time, discomfort, and deleterious side effects. Such techniques were traditionally performed manually via a surgeon manipulating various surgical instruments within the patient's body but can now by implemented using teleoperated robotic systems that provide telepresence. Performing minimally invasive surgery with teleoperated robotic systems facilitates increased precision and range of motion in manipulating surgical instruments when compared to manual techniques, but also introduces new challenges. One such challenge is effective communication between patient-side and remote caregivers in an operating room that often includes many background noises.

### SUMMARY

The following presents a simplified summary of various examples described herein and is not intended to identify key or critical elements or to delineate the scope of the claims.

In accordance with a first example, a medical system is disclosed that includes a communication assembly. The communication assembly includes a cover plate, a circuit board, one or more point light sources, a light guide, a plurality of microphone sets, and a plurality of gaskets. The cover plate has a patient-facing, exterior surface extending along a first direction and includes a plurality of audio openings. The circuit board is coupled to the cover plate and spatially separated from the cover plate by a gap that extends along a second direction transverse to the first direction. The one or more point light sources are electrically coupled to the circuit board and disposed between the circuit board and the cover plate. The light guide is disposed at least partially between the circuit board and the cover plate. The light guide provides at least one light path to direct and diffuse light emitted by the one or more point light sources to the exterior surface, where the at least one light path extends along the first direction and the second direction within the gap. The plurality of microphone sets are electrically coupled to the circuit board and disposed between the circuit board and the cover plate. Each microphone set of the plurality of microphone sets includes a first microphone and a second microphone spaced along the first direction outwardly of the first microphone. Each gasket of the plurality of gaskets includes an audio passageway that extends across the gap from a first seal that seals to one of the first microphone and second microphone to a second seal that seals around an audio opening of the plurality of audio openings.

In some examples, the plurality of microphone sets are disposed in an array having a spoked configuration. In further examples, the plurality of microphone sets can include at least four microphone sets.

In some examples, the plurality of gaskets include gaskets configured for each microphone set. In further examples, each of the gaskets define cavities configured to receive the first microphone and the second microphone.

In some examples, the medical system includes an orienting platform having a patient-facing surface, the exterior surface of the cover plate of the communication assembly exposed along the patient-facing surface of the orienting platform. In further examples, the medical system includes one or more manipulator arms coupled to the orienting platform, a targeting laser for positioning the orienting platform relative to a patient and the cover plate and circuit board define central openings therethrough for the targeting laser, and/or an electromagnetic shield disposed on an opposite side of the circuit board as the cover plate. In further examples, the one or more point light sources include a circular array coupled along a perimeter of the circuit board, the light guide includes an unbroken circular perimeter portion defining radial light paths for the circular array, and the electromagnetic shield comprises an annular gasket disposed rearwardly of the circular perimeter of the light guide for ground path protection.

In some examples, the medical system includes a control system operably coupled to the plurality of microphone sets, where the control system is configured to analyze sound received from the plurality of microphone sets for beam forming. In further examples, the control system is operably coupled to the one or more point light sources and configured to energize the one or more point light sources as a status indicator to a user.

Any of the above examples can further include one or more of the following aspects: the cover plate defines recesses in an interior surface, where the recesses are configured to key the plurality of gaskets relative to the plurality of audio openings; the medical system includes a plurality of screens disposed between the plurality of gaskets and the cover plate, where the plurality of screens are audio transparent and resistant to fluid intrusion; or the one or more point light sources are oriented to emit light in the first direction and the at least one light path of the light guide directs the light from the first direction to the second direction to the exterior surface.

In accordance with a second example, a medical system is disclosed that includes an orienting platform having a patient-facing, distal surface extending along a first direction and a communication assembly extending within the orienting platform along a second direction transverse to the first direction. The communication assembly includes a cover plate, a circuit board, a plurality of microphone sets, a plurality of audio passageways, one or more point light sources, and a light guide. The cover plate has an exterior surface exposed along the distal surface of the orienting platform and defines a plurality of audio openings. The circuit board is spaced proximally from the cover plate along the second direction by a gap. The plurality of microphone sets are electrically coupled to the circuit board and disposed within the gap between the circuit board and the cover plate. Each microphone set of the plurality of microphone sets includes a first microphone and a second microphone spaced from one another in the first direction. The plurality of audio passageways are isolated from each other and each audio passageway extends across the gap from a first seal that seals to one of the first microphone and second microphone to a second seal that seals around an audio opening of the plurality of audio openings defined in the cover plate. The one or more point light sources are electrically coupled to the circuit board and disposed within the gap between the circuit board and the cover plate. The light guide is disposed at least partially within the gap between the circuit board and the cover plate and provides at least one light path to direct and diffuse light emitted by the one or more point light sources to the exterior surface.

In some examples, the plurality of audio passageways are defined by one or more gaskets compressed between the circuit board and the cover plate. In further examples, the plurality of microphone sets are disposed in an array having a spoked configuration and the one or more gaskets comprise a plurality of gaskets for the plurality of microphone sets; the cover plate defines one or more recesses in an interior surface configured to key the one or more gaskets relative to the plurality of audio openings; and/or the one or more gaskets define cavities configured to receive the plurality of microphones.

In any of the above examples, the medical system can include one or more of the following aspects: the one or more point light sources are oriented to emit light in the first direction and the at least one light path of the light guide directs the light from the first direction to the exterior surface; the medical system includes an electromagnetic shield disposed proximally of the circuit board; or the medical system includes a control system operably coupled to the plurality of microphone sets and the one or more point light sources, where the control system configured to analyze sound received from the plurality of microphone sets for beam forming and energize the one or more point light sources as a status indicator to a user.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 is a schematic diagram for a robotically-assisted manipulator system, according to some examples.

FIG. 2A is a schematic diagram of an instrument system according to examples described herein.

FIG. 2B illustrates a distal portion of the instrument system of FIG. 2A with an extended example of an instrument according to examples described herein.

FIG. 3 is a perspective view of a manipulator system according to examples described herein.

FIG. 4 is a top view of a manipulator system according to examples described herein.

FIG. 5A is an exploded perspective view of a communication assembly for a medical system according to examples described herein.

FIG. 5B is a cross-sectional side view of the communication assembly of FIG. 5A according to examples described herein.

FIG. 5C is a cross-sectional side view of a point light source and a portion of a light guide for the communication assembly of FIG. 5A according to examples described herein.

FIG. 5D is a top plan view of a circuit board for the communication assembly of FIG. 5A showing point light sources and microphone sets coupled to the circuit board according to examples described herein.

FIG. 5E is a perspective view of a gasket defining an audio passageway for the communication assembly of FIG. 5A according to examples described herein.

FIG. 5F is a cross-sectional view of the gasket of FIG. 5E according to examples described herein.

FIG. 5G is a top perspective view of a cover plate for the communication assembly of FIG. 5A according to examples described herein.

FIG. 5H is an exploded perspective view of the communication assembly of FIG. 5A including an electromagnetic shield and second circuit board according to examples described herein.

FIG. 5I is a bottom perspective view of an orienting platform having manipulator arms coupled thereto with the communication assembly of FIG. 5A disposed within the orienting platform according to examples described herein.

FIG. 6 is a schematic view of a medical system according to examples described herein.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

In the following description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional. In some instances, well known methods, procedures, components, and circuits have not been described in detail so as not to unnecessarily obscure aspects of the embodiments.

This disclosure describes various instruments and portions of instruments in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (e.g., one or more degrees of rotational freedom such as, roll, pitch, and yaw). As used herein, the term “pose” refers to the position of an object or a portion of an object in at least one degree of translational freedom and to the orientation of that object or portion of the object in at least one degree of rotational freedom (e.g., up to six total degrees of freedom). As used herein, the term “shape” refers to a set of poses, positions, and/or orientations measured along an object. As used herein, the term “distal” refers to a position that is closer to a procedural site and the term “proximal” refers to a position that is further from the procedural site. Accordingly, the distal portion or distal end of an instrument is closer to a procedural site than a proximal portion or proximal end of the instrument when the instrument is being used as designed to perform a procedure.

Medical systems, such as surgical manipulator systems, are typically utilized in operating rooms, which can have a large amount of background noise. Due to this, communication between patient-side and any remote caregivers can be difficult. The systems and methods described herein advantageously include a patient-side communication assembly that has microphone sets that allow for triangulation/beam forming. With this configuration, patient-side voices can be isolated and, in some examples, amplified and broadcasted to remote caregivers, to ensure that communication between patient-side and remote caregivers is preserved regardless of direction of sounds and orientation of the system components.

The communication assembly is suitable for a patient-facing device (e.g., overhead device). For example, the communication assembly is secured from a proximal side with no fasteners through a patient-facing surface, is resistant to cleaning materials and prevents the ingress of fluids into the communication assembly, is not limited by orientation, surgery, or user movement, and is operable without external audio shielding, allowing drapery and other operating room requirements to be positioned as needed.

The communication assembly includes a circuit board and a cover plate, where the circuit board is spaced a distance from the cover plate due to components mounted to the circuit board and operation requirements for those components. For example, the communication assembly includes one or more point light sources and a plurality of microphone sets electrically coupled to the circuit board. A light guide of the interface assembly is at least partially disposed between the cover plate and the circuit board to direct light emitted from the point light sources to an exterior of the interface assembly (e.g., in a patient-facing direction). Due to this configuration, the microphones are spaced from dedicated audio openings defined in the cover plate. To enable the microphones to be utilized for triangulation/beam forming, the interface assembly further includes gaskets that extend between the cover plate and the circuit board to provide isolating audio passageways between the audio openings defined in the cover plate and corresponding microphones.

The medical systems can include an orienting platform and, in some examples, one or more manipulator arms extending from the orienting platform. The orienting platform is movable relative to a patient to be positioned at a desired position and orientation. In these examples, the communication assembly is disposed within a patient-facing surface of the orienting platform. In some examples, the surgical manipulator systems are configured so that the orienting platform is disposed above a patient on a bed, such that a patient-facing surface of the orienting platform is a lower surface of the orienting platform

Aspects of this disclosure herein can be part of a computer-assisted teleoperational manipulator system, sometimes referred to as a robotically-assisted manipulator system or a robotic system. The manipulator system can include one or more manipulators that can be operated with the assistance of an electronic controller (e.g., computer) to move and control functions of one or more instruments when coupled to the manipulators.

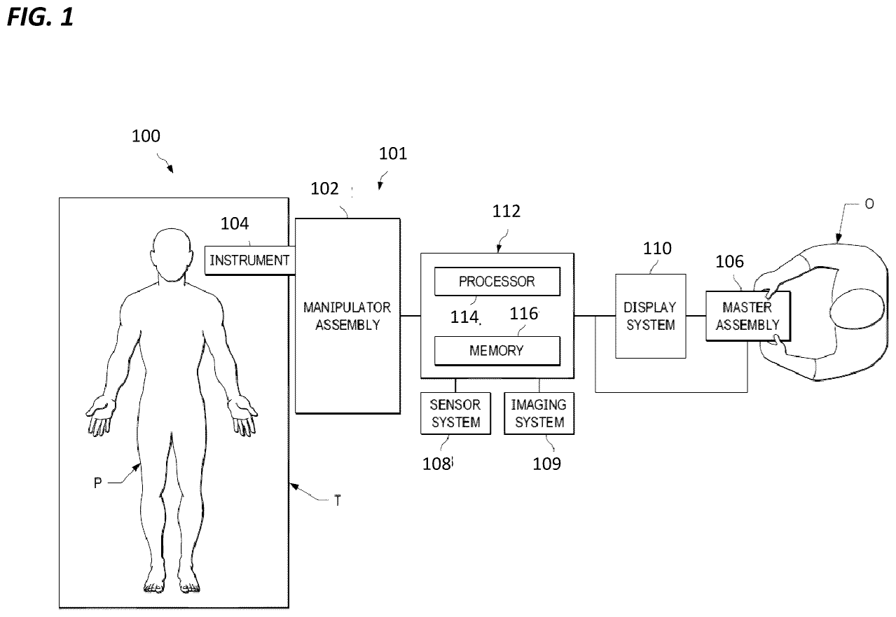

FIG. 1 illustrates an embodiment of a robotically-assisted manipulator system for use with the tools described herein. The manipulator system can be used, for example, in surgical, diagnostic, therapeutic, biopsy, or non-medical procedures, and is generally indicated by the reference numeral 100. As shown in FIG. 1, a robotically-assisted manipulator system 100 can include one or more manipulator assemblies 102 for operating one or more medical instrument systems 104 in performing various procedures on a patient P positioned on a table T in a medical environment 101. For example, the manipulator assembly 102 can drive catheter or end effector motion, can apply treatment to target tissue, and/or can manipulate control members. The manipulator assembly 102 can be teleoperated, non-teleoperated, or a hybrid teleoperated and non-teleoperated assembly with select degrees of freedom of motion that can be motorized and/or teleoperated and select degrees of freedom of motion that can be non-motorized and/or non-teleoperated. An operator input system 106, which can be inside or outside of the medical environment 101, generally includes one or more control devices for controlling manipulator assembly 102. Manipulator assembly 102 supports medical instrument system 104 and can optionally include a plurality of actuators or motors that drive inputs on medical instrument system 104 in response to commands from a control system 112. The actuators can optionally include drive systems that when coupled to medical instrument system 104 can advance medical instrument system 104 into a naturally or surgically created anatomic orifice. Other drive systems can move the distal end of medical instrument in multiple degrees of freedom, which can include three degrees of linear motion (e.g., linear motion along the X, Y, Z Cartesian axes) and in three degrees of rotational motion (e.g., rotation about the X, Y, Z Cartesian axes). The manipulator assembly 102 can support various other systems for irrigation, treatment, or other purposes. Such systems can include fluid systems (including, for example, reservoirs, heating/cooling elements, pumps, and valves), generators, lasers, interrogators, and ablation components.

Robotically-assisted manipulator system 100 also includes a display system 110 for displaying an image or representation of the surgical site and medical instrument system 104 generated by an imaging system 109 which can include an imaging system, such as an endoscopic imaging system. Display system 110 and operator input system 106 can be oriented so an operator O can control medical instrument system 104 and operator input system 106 with the perception of telepresence. A graphical user interface can be displayable on the display system 110 and/or a display system of an independent planning workstation.

In some examples, the endoscopic imaging system components of the imaging system 109 can be integrally or removably coupled to medical instrument system 104. However, in some examples, a separate imaging device, such as an endoscope, attached to a separate manipulator assembly can be used with medical instrument system 104 to image the surgical site. The endoscopic imaging system 109 can be implemented as hardware, firmware, software, or a combination thereof which interact with or are otherwise executed by one or more computer processors, which can include the processors of the control system 112.

Robotically-assisted manipulator system 100 can also include a sensor system 108. The sensor system 108 can include a position/location sensor system (e.g., an actuator encoder or an electromagnetic (EM) sensor system) and/or a shape sensor system (e.g., an optical fiber shape sensor) for determining the position, orientation, speed, velocity, pose, and/or shape of the medical instrument system 104. The sensor system 108 can also include temperature, pressure, force, or contact sensors or the like.

Robotically-assisted manipulator system 100 can also include a control system 112. Control system 112 includes at least one memory 116 and at least one computer processor 114 for effecting control between medical instrument system 104, operator input system 106, sensor system 108, and display system 110. Control system 112 also includes programmed instructions (e.g., a non-transitory machine-readable medium storing the instructions) to implement a procedure using the robotically-assisted manipulator system including for navigation, steering, imaging, engagement feature deployment or retraction, applying treatment to target tissue (e.g., via the application of energy), or the like.

Control system 112 can optionally further include a virtual visualization system to provide navigation assistance to operator O when controlling medical instrument system 104 during an image-guided surgical procedure. Virtual navigation using the virtual visualization system can be based upon reference to an acquired pre-operative or intra-operative dataset of anatomic passageways. The virtual visualization system processes images of the surgical site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like. The control system 112 can use a pre-operative image to locate the target tissue (using vision imaging techniques and/or by receiving user input) and create a pre-operative plan, including an optimal first location for performing treatment. The pre-operative plan can include, for example, a planned size to expand an expandable device, a treatment duration, a treatment temperature, and/or multiple deployment locations.

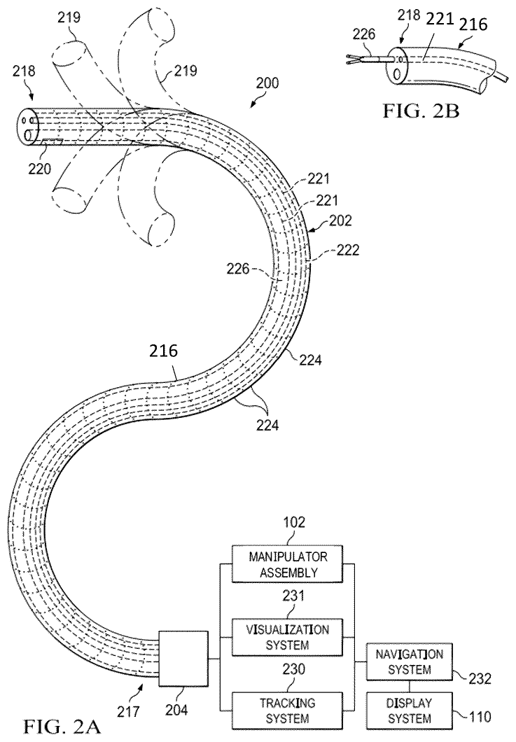

FIG. 2A shows a medical instrument system 200 according to some embodiments. In some embodiments, medical instrument system 200 can be used in an image-guided medical procedure. In some examples, medical instrument system 200 can be used for non-teleoperational exploratory procedures or in procedures involving traditional manually operated medical instruments, such as endoscopy. In some embodiments, medical instrument system 200 is interchangeable with, or a variation of, medical instrument system 104 of FIG. 1.

Medical instrument system 200 includes elongate flexible device 202, such as a flexible catheter or endoscope (e.g., gastroscope, bronchoscope), coupled to a drive unit 204. Elongate flexible device 202 includes a flexible body 216 having proximal end 217 and distal end, or tip portion, 218. In some embodiments, flexible body 216 has an approximately 14-20 mm outer diameter. Other flexible body outer diameters can be larger or smaller. Flexible body 216 can have an appropriate length to reach certain portions of the anatomy, such as the lungs, sinuses, throat, or the upper or lower gastrointestinal region, when flexible body 216 is inserted into a patient's oral or nasal cavity.

Medical instrument system 200 optionally includes a tracking system 230 for determining the position, orientation, speed, velocity, pose, and/or shape of distal end 218 and/or of one or more segments 224 along flexible body 216 using one or more sensors and/or imaging devices. The entire length of flexible body 216, between distal end 218 and proximal end 217, can be effectively divided into segments 224. Tracking system 230 can optionally be implemented as hardware, firmware, software or a combination thereof which interact with or are otherwise executed by one or more computer processors, which can include the processors of control system 112 in FIG. 1.

Tracking system 230 can optionally track distal end 218 and/or one or more of the segments 224 using a shape sensor 222. In some embodiments, tracking system 230 can optionally and/or additionally track distal end 218 using a position sensor system 220, such as an electromagnetic (EM) sensor system. In some examples, position sensor system 220 can be configured and positioned to measure six degrees of freedom, e.g., three position coordinates X, Y, Z and three orientation angles indicating pitch, yaw, and roll of a base point or five degrees of freedom, e.g., three position coordinates X, Y, Z and two orientation angles indicating pitch and yaw of a base point.

Flexible body 216 includes one or more channels 221 sized and shaped to receive one or more medical instruments 226. In some embodiments, flexible body 216 includes two channels 221 for separate instruments 226, however, a different number of channels 221 can be provided.

FIG. 2B is a simplified diagram of flexible body 216 with medical instrument 226 extended according to some embodiments. In some embodiments, medical instrument 226 can be used for procedures and aspects of procedures, such as surgery, biopsy, ablation, mapping, imaging, illumination, irrigation, or suction. Medical instrument 226 can be deployed through channel 221 of flexible body 216 and used at a target location within the anatomy. Medical instrument 226 can include, for example, image capture devices, biopsy instruments, ablation instruments, catheters, laser ablation fibers, and/or other surgical, diagnostic, or therapeutic tools. Medical tools can include end effectors having a single working member such as a scalpel, a blunt blade, a lens, an optical fiber, an electrode, and/or the like. Other end effectors can include, for example, forceps, graspers, balloons, needles, scissors, clip appliers, and/or the like. Other end effectors can further include electrically activated end effectors such as electrosurgical electrodes, transducers, sensors, imaging devices and/or the like. Medical instrument 226 can be advanced from the opening of channel 221 to perform the procedure and then retracted back into the channel when the procedure is complete. Medical instrument 226 can be removed from proximal end 217 of flexible body 216 or from another optional instrument port (not shown) along flexible body 216. The medical instrument 226 can be used with an image capture device (e.g., an endoscopic camera) also within the elongate flexible device 202. Alternatively, the medical instrument 226 can itself be the image capture device.

Medical instrument 226 can additionally house cables, linkages, or other actuation controls (not shown) that extend between its proximal and distal ends to controllably the bend distal end of medical instrument 226. Flexible body 216 can also house cables, linkages, or other steering controls (not shown) that extend between drive unit 204 and distal end 218 to controllably bend distal end 218 as shown, for example, by broken dashed line depictions 219 of distal end 218. In some examples, at least four cables are used to provide independent “up-down” steering to control a pitch motion of distal end 218 and “left-right” steering to control a yaw motion of distal end 218. In embodiments in which medical instrument system 200 is actuated by a robotically-assisted assembly, drive unit 204 can include drive inputs that removably couple to and receive power from drive elements, such as actuators, of the teleoperational assembly. In some embodiments, medical instrument system 200 can include gripping features, manual actuators, or other components for manually controlling the motion of medical instrument system 200. The information from tracking system 230 can be sent to a navigation system 232 where it is combined with information from visualization system 231 and/or the preoperatively obtained models to provide the physician or other operator with real-time position information.

Other configurations of teleoperated manipulator systems are also contemplated, such as systems configured for multi-port or single-port procedures. For example, the embodiments described herein can be used with a da Vinci® Surgical System, such as the da Vinci X®, Xi®, or SP® Surgical Systems, all commercialized by Intuitive Surgical, Inc., of Sunnyvale, California.

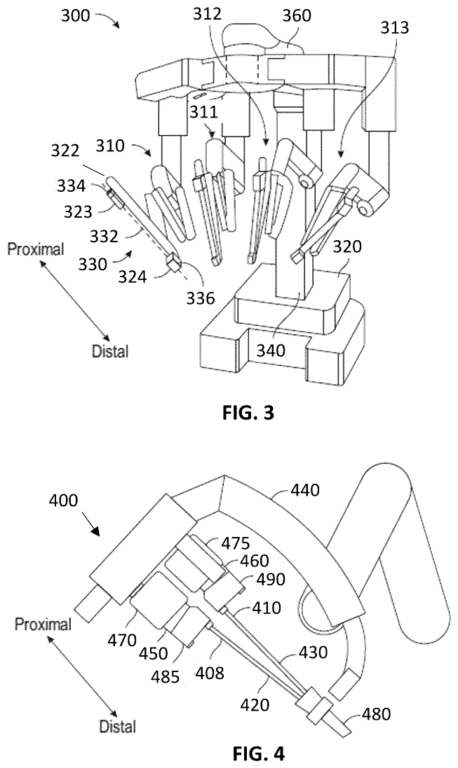

FIG. 3 illustrates an example embodiment of a manipulator system 300 that can be used as part of the manipulator system 100. The manipulator system 300 includes a base 320, a main column 340, and a main boom 360 connected to main column 340. Manipulator system 300 also includes a plurality of manipulator arms 310, 311, 312, 313, which are each connected to main boom 360. The manipulator arms 310, 311, 312, and 313, can be used as the manipulator assemblies 102. Manipulator arms 310, 311, 312, 313 each include an instrument mount portion 322 to which an instrument 330 can be mounted, which is illustrated as being attached to manipulator arm 310. While the manipulator system 300 depicts four manipulator arms, various embodiments can include more or fewer manipulator arms.

Instrument mount portion 322 can include a drive assembly 323 and a cannula mount 324, with a transmission mechanism 334 of the instrument 330 connecting with the drive assembly 323, according to an embodiment. Cannula mount 324 is configured to hold a cannula 336 through which a shaft 332 of instrument 330 can extend to a surgery site during a surgical procedure. Drive assembly 323 contains a variety of drive and other mechanisms that are controlled to respond to input commands at the operator input system 106 and transmit forces to the transmission mechanism 334 to actuate the instrument 330. Although the embodiment of FIG. 3 shows an instrument 330 attached to only manipulator arm 310 for case of viewing, an instrument can be attached to any and each of manipulator arms 310, 311, 312, 313.

FIG. 4 illustrates an example embodiment of a manipulator system 400 that can be used as part of the manipulator system 100. In FIG. 4, a portion of a manipulator arm 440 of the manipulator system 400 is shown with two instruments 408, 410 in an installed position. The schematic illustration of FIG. 4 depicts only two instruments for simplicity, but more than two instruments can be mounted in an installed position at the manipulator system 400 as those having ordinary skill in the art are familiar. Each instrument 408, 410 includes a shaft 420, 430 having at a distal end a moveable end effector or an endoscope, camera, or other sensing device, and can or cannot include a wrist mechanism (not shown) to control the movement of the distal end.

In the embodiment of FIG. 4, the distal end portions of the instruments 408, 410 are received through a single port structure 480 to be introduced into the patient. As shown, the port structure includes a cannula and an instrument entry guide inserted into the cannula. Individual instruments are inserted into the entry guide to reach a surgical site.

Transmission mechanisms 485, 490 are disposed at a proximal end portion of each shaft 420, 430 and connect through a sterile adaptor 450, 460 with drive assemblies 470, 475, which contain a variety of internal mechanisms (not shown) that are controlled by a controller (e.g., at a control cart of a surgical system) to respond to input commands at a surgeon side console of a surgical system to transmit forces to the force transmission mechanisms 485, 490 to actuate instruments 408, 410.

The manipulator systems described herein are not limited to the embodiments of FIGS. 1, 2, 3, and 4, and various other teleoperated, computer-assisted manipulator configurations can be used with the embodiments described herein. The diameter or diameters of an instrument shaft and end effector are generally selected according to the size of the cannula with which the instrument will be used and depending on the surgical procedures being performed.

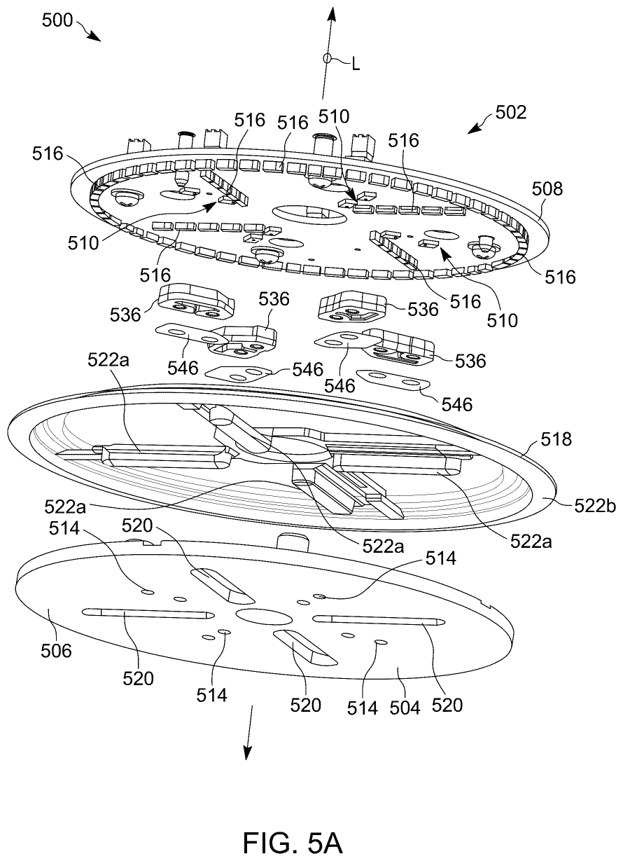

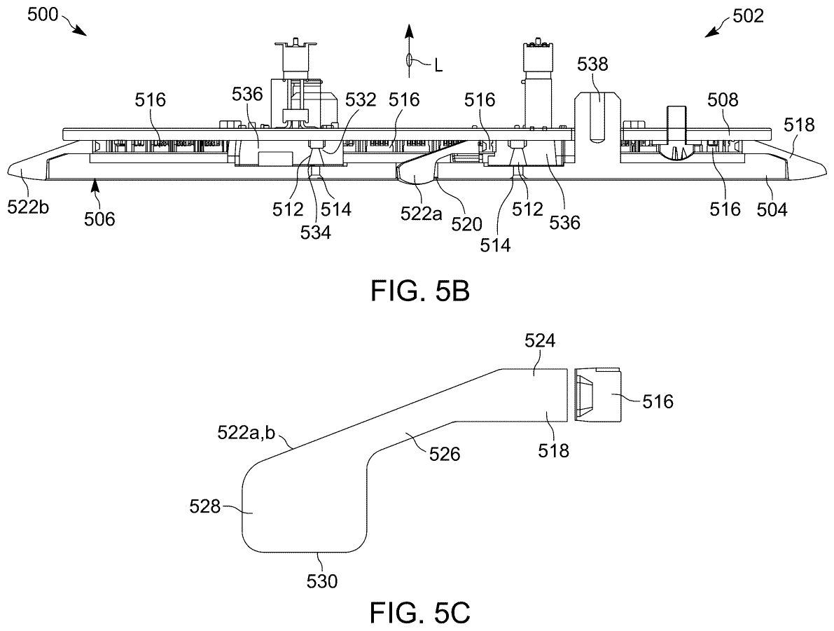

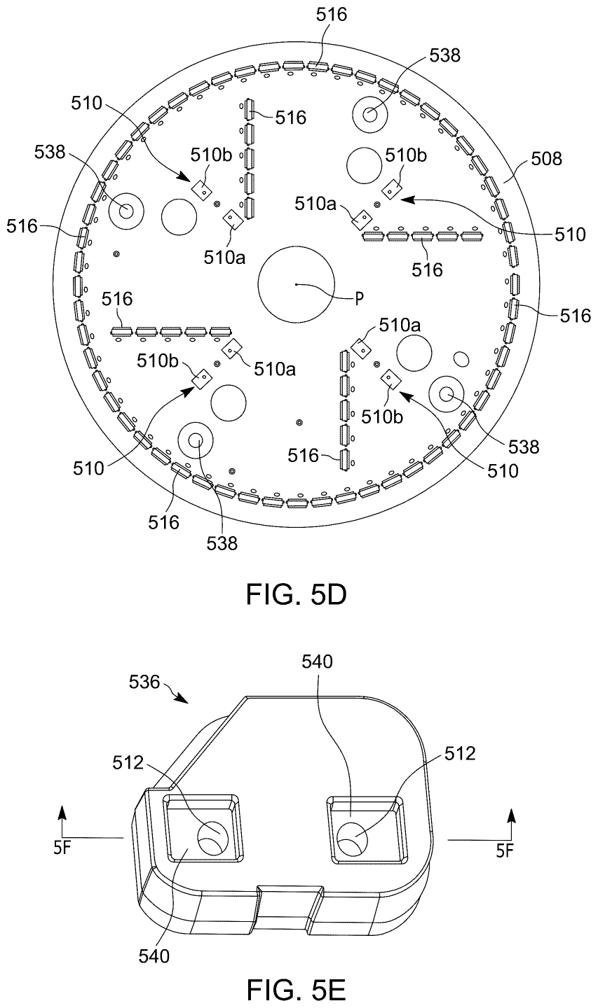

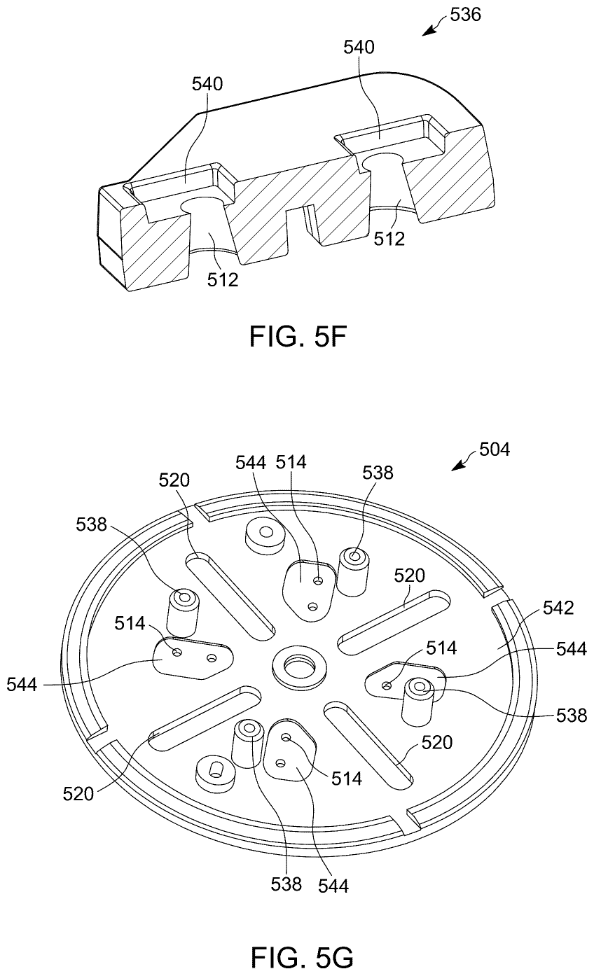

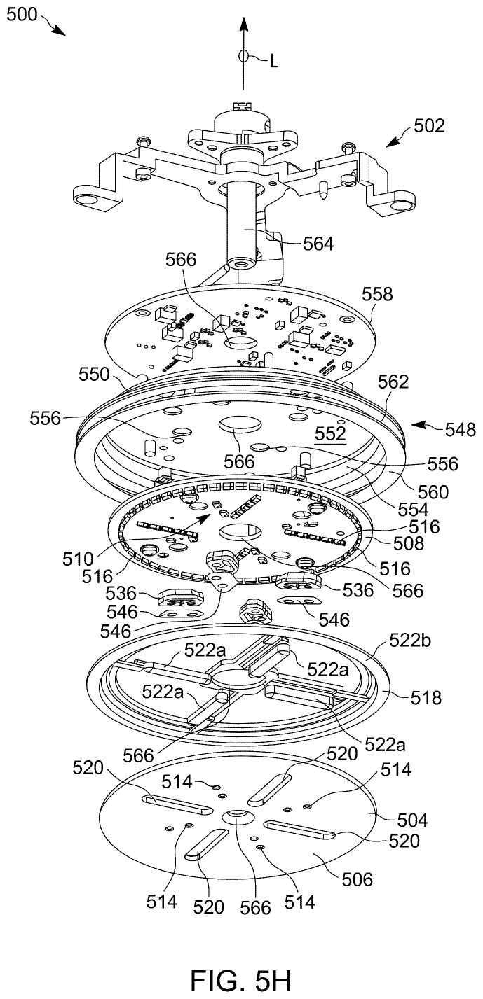

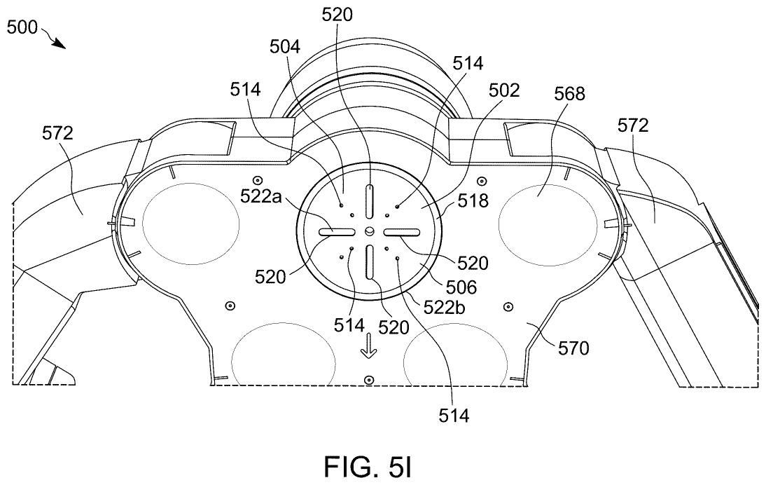

A medical system 500, such as a surgical manipulator system, is shown in FIGS. 5A-5H. According to some examples consistent with FIGS. 1-4, the medical system 500 may correspond to, or be incorporated into, the manipulator system 100, medical instrument system 200, the manipulator system 300, and/or the manipulator system 400 described above.

As shown in the figures, the medical system 500 includes a communication assembly 502 suitable for isolating voices patient-side and relaying the sound to remote users, such as users at other carts or consoles of the medical system 500. The communication assembly 502 includes components or portions of components, described in more detail below, extending a first direction and assembled/stacked along a second direction transverse to the first direction. The second direction extends from a proximal, patient side end to an opposite, distal end. Within the frame of reference of the communication assembly 502, the second direction can be considered to extend along a longitudinal axis L with components or portions of components of the communication assembly 502 extending transversely to the longitudinal axis L in the first direction. It will be understood that “traverse” when used herein with reference to two objects can include the two objects extending at an angle relative to one another. For example, the objects can be orthogonal with respect to one another, at an angle relative to another in a range of between 0 and 3 degrees, between 0 and 5 degrees, between 0 and 10 degrees, between 0 and 15 degrees, and so forth.

The communication assembly 502 includes a cover plate 504 having a patient-facing, exterior surface 506 and a circuit board 508 coupled to the cover plate 504 and spatially separated from the cover plate 504 by a gap that extends along the second direction (e.g., spaced proximally from the cover plate 504 along the longitudinal axis). The communication assembly 502 further includes a plurality of microphone sets 510 electrically coupled to the circuit board 508 and disposed between the circuit board 508 and the cover plate 504 (e.g., disposed within the gap between the circuit board 508 and the cover plate 504). Each microphone in each microphone set 510 has an associated isolated audio passageway 512 (FIGS. 5D-5F) extending across the gap between one of a plurality of audio openings 514 defined in the cover plate 504 and the respective microphone. Each microphone set 510 includes a first microphone 510a and a second microphone 510b spaced from one another to provide audio input for beam forming/triangulation analysis. For example, the second microphone 510b is spaced along the first direction outwardly of the first microphone 510a, the first and second microphones 510a, 510b are spaced from one another in the first direction transverse to the second direction, and/or the first and second microphones 510a, 510b are spaced radially from one another.

The communication assembly 502 further includes one or more point light sources 516 electrically coupled to the circuit board 508 and disposed between the circuit board 508 and the cover plate 504 (e.g., disposed within the gap between the circuit board 508 and the cover plate 504) and a light guide 518 disposed at least partially between the circuit board 508 and the cover plate 504 (e.g., within the gap) that has at least one light path to direct and diffuse light emitted by the one or more point light sources 516 to the exterior surface 506. In some examples, the at least one light path extends along the first direction and the second direction within the gap.

In some examples, the point light sources 516 and the light guide 518 are configured to emit light in one or more locations spaced inwardly from an edge of the cover plate 504. As such, in these examples, the cover plate 504 includes openings 520 extending therethrough that are configured to receive a portion of the light guide 518 at least partially therein.

In some implementations, it can be desirable to diffuse light emitted by the point light sources 516 so that light emitted through the light guide 518 has a uniform intensity and color. As discussed in more detail below, keeping the distance between the cover plate 504 and the circuit board 508 as small as possible advantageously helps sound isolation the subsequent beam forming processing. Accordingly, to ensure that light emitted from the point light sources 516 has enough travel for sufficient diffusion, the point light sources 516 can be coupled to the circuit board 508 to emit light in the first direction (e.g., transverse to the longitudinal axis) and the light guide 518 can be configured to provide a light path that directs the emitted light from the first direction to the second direction to the exterior surface 506.

As shown in FIGS. 5B-5D, the light guide 518 includes interior and exterior portions 522a, 522b. The interior portions 522a are configured to direct light emitting from the point light sources 516 through the openings 520 of the cover plate 504, while the exterior portions 522b are configured to direct light along some or all of the edge of the cover plate 504. For example, as shown, the exterior portions 522b can be an unbroken portion of the light guide 518 extending around an entire edge (e.g., circumference) of the cover plate 504 to define radial light paths for the point light sources 516. The point light sources 516 include point light sources 516 aligned with the interior and exterior portions 522a, 522b of the light guide 518. For example, the point light sources 516 can include point light sources 516 disposed in an array along a perimeter of the circuit board 508, where the array has a shape corresponding to the perimeter (e.g., circular). The point light sources 516 can also be disposed in lines (e.g., straight and/or curved alignment) across an interior of the circuit board 508. As shown in FIG. 5D, the point light sources 516 include four line arrays spaced around the circuit board 508. The line arrays are offset from two perpendicular lines extending through the center of the circuit board 508 and oriented toward the adjacent line, so that with the light guide 518, the communication assembly 502 has four interior portions 522a arrayed in a spoked configuration equally around the point P.

As shown in FIG. 5C, both light guide portions 522a, 522b include a light entrance 524 with a surface oriented (e.g., extending generally along the second direction) to receive light from the point light sources 516 emitted along the first direction, an intermediate neck portion 526, and a distal head portion 528. The neck portion 526 has a relatively narrowed depth relative to the head portion 528 and is angled downwardly relative to the first direction, which causes light emitted from the point light sources 516 to be repeatedly reflected within the neck portion 526 for diffusion. With this configuration, the depth of the neck portion 526 and the lateral length of the interior/exterior portion 522a, 522b along the first direction, including the lateral length of the neck portion 526, can advantageously be configured to produce diffuse light within a minimized vertical space due to the audio constraints discussed in more detail below.

The head portion 528 includes a downward surface 530 that, when the communication assembly 502 is assembled, is exposed along and adjacent to the exterior surface 506 of the cover plate 504. The light guide 518 is configured so that light emitted from the point light sources 516 is directed along the light path through the downward surface 530 generally toward a patient. As shown, the downward surface 530 generally extends in the first direction. It will be understood that generally extending in the first direction can include a convex curvature as shown in FIG. 5B, including a curvature symmetrical relative to the second direction as shown by the interior portions 522a or curved upwardly as shown by the exterior portions 522b. Alternatively, one or both of the interior portions 522a and exterior portions 522b can have a flat downward surface 530 as shown in FIG. 5C.

The audio passageways 512 of the communication assembly 502 are air/sound tight so that the communication assembly 502 is able to provide isolated inputs for each microphone. Pursuant to this, each audio passageway 512 extends across the gap from a first seal 532 that seals to one of the microphones 510a, 510b to a second seal 534 that seals to the cover plate 504 around the associated audio opening 514. As shown in FIGS. 5B, 5E, and 5F, the audio passageways 512 are defined by one or more gaskets 536 disposed between the cover plate 504 and the circuit board 508. The gaskets 536 are sized to at least partially compress between the cover plate 504 and the circuit board 508 when the cover plate 504 and circuit board 508 are secured together during assembly of the communication assembly 502 to form the first and second seals 532, 534. In some examples, the communication assembly 502 can be configured so that the microphone sets 510 are disposed closely adjacent to one of a plurality of fastener openings 538 that receive a fastener therein to secure the components of the communication assembly 502 together. With this configuration, the fastener tightening the components of the communication assembly 502 fully together, including the cover plate 504 and circuit board 508, ensures that a sufficient compressive force is applied to the gaskets 536.

As shown in FIG. 5B, the gasket 536 defines the audio passageway 512 through a height thereof. The audio passageway 512 can have a cylindrical configuration or a frustoconical configuration as shown. For example, the gasket 536 can be disposed between the cover plate 504 and the circuit board 508 so that the larger diameter end of the frustoconical configuration is disposed around the audio opening 514, while the smaller diameter end of the frustoconical configuration is aligned with an input of the microphone 510a, 510b. In some examples, the audio passageway 512 is configured to be sufficiently large to not alter sound between the audio opening 514 and the microphone 510a, 510b across the voice spectrum, but also not large enough to create a cavity that may distort sound traveling through the passageway 512.

To help with assembly, the communication assembly 502 can include alignment features to ensure that the gaskets 536 are correctly positioned relative to the microphone 510a, 510b and the audio opening 514 to provide the audio passageway 512. In one example, the gasket 536 can define a cavity 540 sized to receive the microphone 510a, 510b therein. The cavity 540 allows the material of the gasket 536 to extend around the sides of the microphone housing, such that with the microphone 510a, 510b coupled to the circuit board 508, the gasket 536 is held in place relative to the circuit board 508. In an additional example, an interior surface 542 of the cover plate 504 defines one or more recesses 544 sized to receive the gaskets 536 therein. The recesses 544 have a shape corresponding to a shape of the gaskets 536, so that the gaskets 536 are keyed in place relative to the audio openings 514 and held in place relative to the cover plate 504.

The gaskets 536 can be configured for individual microphones 510a, 510b in the microphone set 510, such that each microphone 510a, 510b has a dedicated gasket 536. In some examples, the gaskets 536 can be sized and configured to define audio passageways 512 for two or more microphones 510a, 510b. For example, the communication assembly 502 can include one gasket 536 for each microphone set 510. Pursuant to this, each gasket 536 can include spaced cavities 538 for the first and second microphones 510a, 510b.

Although a single microphone set 510 is suitable for some implementations, the medical system 500 can alternatively include two, three, four, or more microphone sets 510 to provide a desired number of inputs for beam forming/triangulation analysis.

In some examples, the microphone sets 510 are arrayed about a point P (FIG. 5D) of the communication assembly 502. For example, the point P can be a longitudinal center point of the communication assembly 502. With this configuration, the first and second microphones 510a, 510b of each microphone set 510 are radially spaced from the point P and form one another relative to the point P. Further, the microphone sets 510 are spaced circumferentially from one another about the point P. As such, this arrays the microphone sets 510 in a spoked configuration. As shown in FIG. 5D, the communication assembly 502 can include four spaced microphone sets 510 equally arrayed around the point P and with the first and second microphones 510a, 510b of each microphone set 510 radially aligned and spaced from one another.

As shown in FIG. 5A, the communication assembly 502 can include one or more screens 546 that are disposed between the audio opening 514 and the microphone 510a, 510b. The screens 546 are audio transparent and resistant to fluid intrusion to protect the microphones 510a, 510b against damage from fluids. Further, the screens 546 can be disposed between the gaskets 536 and the cover plate 504 and held in place both by the cavities 540 and the compression of the gaskets 536 during assembly to prevent the ingress of fluids past the audio opening 514. In some examples, the communication assembly 502 includes as many screens 546 as gaskets 536, with the screens 546 having a similar shape to a perimeter of the gaskets 536. Pursuant to this, the screens 546 can be configured to extend over multiple microphones 510a, 510b, such as a screen 546 for each microphone set 510.

In some implementations as shown in FIG. 5H, the communication assembly 502 further includes an electromagnetic shield 548 to protect electronics disposed proximally of the circuit board 508 from damage, such as that due to electrostatic discharge. The electromagnetic shield 548 includes a shield plate 550 having a main wall 552 that extends along the first direction and a skirt 554 that depends distally from edges of the main wall 552, such that the skirt 554 is disposed outwardly (e.g., radially outwardly) of the circuit board 508 and the components coupled thereto (e.g., the microphone sets 510 and point light sources 516) when the communication assembly 502 is fully assembled.

As shown, the main wall 552 can include a plurality of openings 556 extending therethrough to allow for electronic connections and other components to extend along the second direction through the communication assembly 502. Pursuant to this, the communication assembly 502 of this example includes a second circuit board 558 disposed proximally of the electromagnetic shield plate 550, which carries circuitry susceptible to damage, such as due to electrostatic discharge.

In implementations where the point light sources 516 and the light guide 518 result in an unbroken exterior portion 522b for the light guide 518, the electromagnetic shield 548 further includes a gasket 560 having a shape corresponding to the light guide exterior portion 522b. Optionally, the shield plate 550 can include a flange 562 extending outwardly from a distal edge of the skirt 554. The flange 562 and gasket 560 can have a similar shape (e.g., annular) to couple together when the components of the communication assembly 502 are assembled together. The gasket 560 provides ground path protection for the communication assembly 502 around the unbroken light guide exterior portion 522b.

As shown in FIG. 5H, the communication assembly 502 further includes a targeting laser 564 that is disposed within and aligned with openings 566 extending through the cover plate 504, light guide 518, circuit board 508, shield plate 550, and second circuit board 558 for positioning the communication assembly 502 relative to a patient.

In the example shown in FIG. 5I, in addition to the communication assembly 502, the medical system 502 further includes an orienting platform 568 having a patient-facing, distal surface 570. The exterior surface 506 of the cover plate 504 of the communication assembly 502 is exposed along the patient-facing surface 570 of the orienting platform 568, such that the communication assembly 502 extends within the orienting platform 568 along the second direction. With this configuration, a user manipulates the position and orientation of the orienting platform 568 relative to a patient. In some examples, the medical system 502 further includes one or more manipulator arms 572 coupled to the orienting platform 568. For medical systems 502 consistent with FIGS. 3 and/or 4, the manipulator arms 572 can correspond to the manipulator arms 310, 311, 312, 313, 440 described above.

In some examples, the communication assembly 502 is configured to isolate sounds in a range of about 10 Hz to about 12 kHz, or in a range of about 150 Hz to about 6 kHz. In some examples, the point light sources 516 can be RGBW light emitting diodes. The point light sources 516 can be individually controlled. Any suitable number of point light sources 516 can be utilized, including up to 100 light sources or seventy six light sources as shown in FIG. 5D. In some examples, the light guide 518 can be formed from polyethylene terephthalate glycol (PETG). In some examples, the microphones 510a, 510b can be MEMS audio sensor omnidirectional digital microphones. In some examples, the audio openings 514 can be about 3 mm. In some examples, the screens 546 can be made at least partially from Saatifil Acoustex or other material that is acoustically neutral and prevents the passage of fluids.

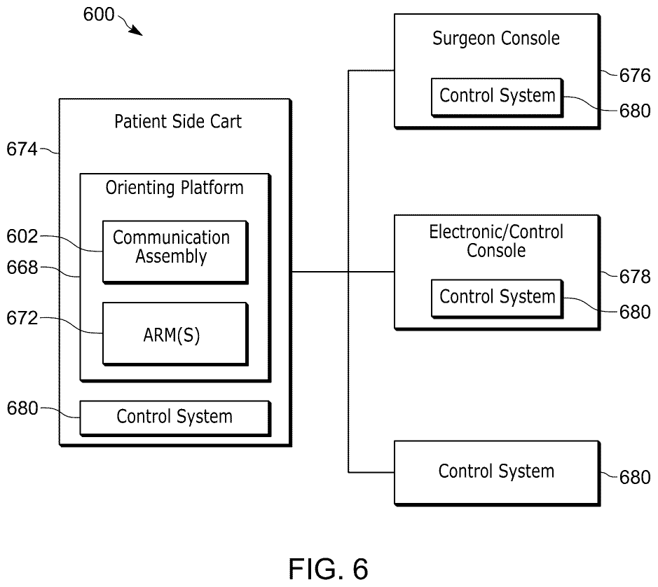

An example medical system 600 (e.g., a minimally invasive robotic surgical system) is shown in FIG. 6 that includes a communication assembly 602 (e.g., communication assembly 502) received within an orienting platform 668 (e.g., orienting platform 568). The medical system 600 further includes a patient side cart 674, a surgeon console 676, and an electronics/control console 678. It is noted that the components diagrammatically shown in FIG. 6 are not shown in any particular positioning and can be arranged as desired, with the patient side cart 674 being disposed relative to the patient so as to effect surgery on the patient.

The medical system 600 is used to perform minimally invasive robotic surgery by interfacing with and controlling a variety of surgical instruments, as those of ordinary skill in the art are generally familiar. The patient side cart 674 includes the orienting platform 668 and one or more jointed arms 672 (e.g., manipulator arms 572) for holding, positioning, and manipulating various tools, including, but not limited to, for example, a surgical instrument with an end effector and an endoscope (not shown). The patient side cart 674 is positioned proximate to a patient and one or more surgical instruments are used to perform various surgical procedures at a work site in the patient's body. Exemplary surgical procedures that an end effector can perform include, but are not limited to, for example, clamping of a vessel or other hollow body structure, cutting tissue using pivoting blades of an end effector, and/or other procedures where a relatively high gripping force may be desirable.

In general, the surgeon console 676 receives inputs from a surgeon by various input devices and serves as a master controller by which the patient side cart 674 acts as a slave to implement the desired motions of the surgical instrument(s) interfaced therewith, and accordingly perform the desired surgical procedure.

The electronics/control console 678, which may include, for example, an electrosurgical processing unit, receives and transmits various control signals to and from the patient side cart 674 and the surgeon console 676, and can transmit light and process images (e.g., from an endoscope at the patient side cart 674) for display, such as, for example, a display (not shown) at the surgeon console 676 and/or associated with the electronics/control console 678. Those having ordinary skill in the art are generally familiar with such electronics/control consoles of robotically controlled surgical systems.

An electronic data processing system, including a control system 680, and which may be provided at one or more of the patient side cart 674, the surgeon console 676, and the electronics/control console 678, may receive and process inputs from the surgeon console 676 and control the manipulation of the arms 672 and instruments coupled thereto at the patient side cart 674 based on the inputs received at the surgeon console 676. However, the present disclosure is not limited to receiving inputs at the surgeon console 676, and inputs may be received at any device which results in manipulation of components of the patient side cart 674 (e.g., the communication assembly 602, the arms 672, and/or instruments/end effectors coupled to the arms 672).

The communication assembly 602 of this implementation can correspond to the communication assembly 502 described above and include any of the components thereof. As such, the control system 680 is operably coupled to a plurality of microphone sets 610 and configured to analyze sound received from the plurality of microphone sets 610 for beam forming. Further, the control system 680 is operably coupled to one or more point light sources 616 and configured to energize the one or more point light sources 616 as a status indicator (e.g., fault, operational, processing, etc.) to a user.

One or more components of the embodiments discussed in this disclosure, such as control system 112, 680, may be implemented in software for execution on one or more processors of a computer system. The software may include code that when executed by the one or more processors, configures the one or more processors to perform various functionalities as discussed herein. The code may be stored in a non-transitory computer readable storage medium (e.g., a memory, magnetic storage, optical storage, solid-state storage, etc.). The computer readable storage medium may be part of a computer readable storage device, such as an electronic circuit, a semiconductor device, a semiconductor memory device, a read only memory (ROM), a flash memory, an erasable programmable read only memory (EPROM); a floppy diskette, a CD-ROM, an optical disk, a hard disk, or other storage device. The code may be downloaded via computer networks such as the Internet, Intranet, etc. for storage on the computer readable storage medium. The code may be executed by any of a wide variety of centralized or distributed data processing architectures. The programmed instructions of the code may be implemented as a number of separate programs or subroutines, or they may be integrated into a number of other aspects of the systems described herein. The components of the computing systems discussed herein may be connected using wired and/or wireless connections. In some examples, the wireless connections may use wireless communication protocols such as Bluetooth, near-field communication (NFC), Infrared Data Association (IrDA), home radio frequency (HomeRF), IEEE 802.11, Digital Enhanced Cordless Telecommunications (DECT), and wireless medical telemetry service (WMTS).

Various general-purpose computer systems may be used to perform one or more processes, methods, or functionalities described herein. Additionally or alternatively, various specialized computer systems may be used to perform one or more processes, methods, or functionalities described herein. In addition, a variety of programming languages may be used to implement one or more of the processes, methods, or functionalities described herein.

While certain embodiments and examples have been described above and shown in the accompanying drawings, it is to be understood that such embodiments and examples are merely illustrative and are not limited to the specific constructions and arrangements shown and described, since various other alternatives, modifications, and equivalents will be appreciated by those with ordinary skill in the art.

1. A medical system comprising:
a communication assembly comprising:
a cover plate having a patient-facing, exterior surface extending along a first direction, the cover plate comprising a plurality of audio openings;
a circuit board coupled to the cover plate and spatially separated from the cover plate by a gap that extends along a second direction;
one or more point light sources electrically coupled to the circuit board and disposed between the circuit board and the cover plate;
a light guide disposed at least partially between the circuit board and the cover plate, the light guide comprising at least one light path to direct and diffuse light emitted by the one or more point light sources to the exterior surface, the at least one light path extending along the first direction and the second direction within the gap;
a plurality of microphone sets electrically coupled to the circuit board and disposed between the circuit board and the cover plate, each microphone set of the plurality of microphone sets including a first microphone and a second microphone spaced along the first direction outwardly of the first microphone; and
a plurality of gaskets, each gasket of the plurality of gaskets comprising an audio passageway extending across the gap from a first seal that seals to one of the first microphone and second microphone to a second seal that seals around an audio opening of the plurality of audio openings.

2. The medical system of claim 1, wherein the plurality of microphone sets are disposed in an array having a spoked configuration.

3. The medical system of claim 2, wherein the plurality of microphone sets comprise at least four microphone sets.

4. The medical system of claim 1, wherein the plurality of gaskets comprise gaskets configured for each microphone set.

5. The medical system of claim 4, wherein each of the gaskets define cavities configured to receive the first microphone and the second microphone.

6. The medical system of claim 1, wherein the cover plate defines recesses in an interior surface, the recesses configured to key the plurality of gaskets relative to the plurality of audio openings.

7. The medical system of claim 1, further comprising a plurality of screens disposed between the plurality of gaskets and the cover plate, the plurality of screens being audio transparent and resistant to fluid intrusion.

8. The medical system of claim 1, wherein the one or more point light sources are oriented to emit light in the first direction; and the at least one light path of the light guide directs the light from the first direction to the second direction to the exterior surface.

9. The medical system of claim 1, further comprising an orienting platform having a patient-facing surface, the exterior surface of the cover plate of the communication assembly exposed along the patient-facing surface of the orienting platform.

10. The medical system of claim 9, further comprising one or more manipulator arms coupled to the orienting platform.

11. The medical system of claim 9, further comprising an electromagnetic shield disposed on an opposite side of the circuit board as the cover plate.

12. The medical system of claim 11, wherein the one or more point light sources include a circular array coupled along a perimeter of the circuit board; the light guide includes an unbroken circular perimeter portion defining radial light paths for the circular array; and the electromagnetic shield comprises an annular gasket disposed rearwardly of the circular perimeter of the light guide for ground path protection.

13. The medical system of claim 9, further comprising a targeting laser for positioning the orienting platform relative to a patient; and wherein the cover plate and circuit board define central openings therethrough for the targeting laser.

14. The medical system of claim 1, further comprising a control system operably coupled to the plurality of microphone sets, the control system configured to analyze sound received from the plurality of microphone sets for beam forming.

15. The medical system of claim 14, wherein the control system is further operably coupled to the one or more point light sources, the control system configured to energize the one or more point light sources as a status indicator to a user.

16. A medical system comprising:
an orienting platform having a patient-facing, distal surface extending along a first direction;
a communication assembly extending within the orienting platform along a second direction transverse to the first direction, the communication assembly comprising:
a cover plate having an exterior surface exposed along the distal surface of the orienting platform, the cover plate defining a plurality of audio openings;
a circuit board spaced proximally from the cover plate along the second direction by a gap;
a plurality of microphone sets electrically coupled to the circuit board and disposed within the gap between the circuit board and the cover plate, each microphone set of the plurality of microphone sets including a first microphone and a second microphone spaced from one another in the first direction;
a plurality of audio passageways isolated from each other, each audio passageway extending across the gap from a first seal that seals to one of the first microphone and second microphone to a second seal that seals around an audio opening of the plurality of audio openings defined in the cover plate;
one or more point light sources electrically coupled to the circuit board and disposed within the gap between the circuit board and the cover plate; and
a light guide disposed at least partially within the gap between the circuit board and the cover plate, the light guide comprising at least one light path to direct and diffuse light emitted by the one or more point light sources to the exterior surface.

17. The medical system of claim 16, wherein the plurality of audio passageways are defined by one or more gaskets compressed between the circuit board and the cover plate.

18. The medical system of claim 17, wherein the plurality of microphone sets are disposed in an array having a spoked configuration; and the one or more gaskets comprise a plurality of gaskets for the plurality of microphone sets.

19. The medical system of claim 17, wherein the cover plate defines one or more recesses in an interior surface configured to key the one or more gaskets relative to the plurality of audio openings; and the one or more gaskets define cavities configured to receive the plurality of microphones.

20. The medical system of claim 16, wherein the one or more point light sources are oriented to emit light in the first direction; and the at least one light path of the light guide directs the light from the first direction to the exterior surface.

21. The medical system of claim 16, further comprising an electromagnetic shield disposed proximally of the circuit board.

22. The medical system of claim 16, further comprising a control system operably coupled to the plurality of microphone sets and the one or more point light sources, the control system configured to:
analyze sound received from the plurality of microphone sets for beam forming; and
energize the one or more point light sources as a status indicator to a user.
